(19) United States
(12) Patent Application Publication
(10) Pub. No.: US 2005/0221349 A1
(43) Pub. Date: Oct. 6, 2005

(54) Methods of detecting target molecules and molecular interactions

(76) Inventors: Stuart Wilson, London (GB); Christopher John Stanley, Cambridge (GB)

Correspondence Address: FOLEY & LARDNER LLP, 111 HUNTINGTON AVENUE, 26TH FLOOR, BOSTON, MA, 02199-7610, US

(21) Appl. No.: 10/998,503

(22) Filed: Nov. 29, 2004

Related U.S. Application Data
(63) Continuation of application No. PCT/GB03/02383, filed on May 30, 2003.

(30) Foreign Application Priority Data
May 30, 2002 (GB) 0212544.1

(51) Int. Cl. C12Q 1/68; G01N 33/53; G01N 33/537; G01N 33/543

(52) U.S. Cl. 435/6; 435/7.92

(57) ABSTRACT

Methods and kits for detecting target molecules or for monitoring interactions between target molecules. In particular, the invention relates to methods based on the use of “two-component conjugate” systems comprising a catalytic label and a substrate label.

### RELATED APPLICATIONS

This invention is a Continuation of International patent application number PCT/GB03/02383 filed May 30, 2003, and claims the benefit of priority to Great Britain patent application number 0212544.1 filed May 30, 2002, which are incorporated by reference herein in their entireties.

### FIELD OF THE INVENTION

The invention relates to methods and kits for detecting target molecules or the interactions between target molecules. In particular, the invention relates to methods based on the use of “two-component conjugate” systems comprising a catalytic label and a substrate label.

### BACKGROUND TO THE INVENTION

Detection of a target biological molecule in a sample requires sensitive techniques that are capable of discriminating between specific recognition events and non-specific recognition events that might otherwise lead to false positive results.

Techniques for detecting many different types of biological molecules, including proteins, nucleic acids, viruses, bacteria and carbohydrates, are known in the art. High specificity of detection is often achieved by using multiple binding entities to bind to the target molecule that is to be detected.

Known techniques for the detection of nucleic acids include, for example, the Polymerase Chain Reaction (PCR). In this technique two short nucleic acid primers recognise the target nucleic acid. Detection of the target nucleic acid is only achieved when both primers are bound to, and linked through, the same target molecule. Non-specific interactions of the primers with other molecules are not detected unless both primers bind to and are linked by this non-specific interaction. The conditions of the reaction are such that the latter is highly unlikely. Other molecular amplification methods known in the art include nucleic acid sequence-based amplification (NASBA) (Compton, 1991) and 3SR (Fahy et al., 1991).

Proteins may be detected by well known techniques such as Western Blotting. This technique is very useful but is limited in sensitivity and specificity, due to the lack of an amplification step, and the fact that there is a single entity binding to the target.

Target molecules can, however, be detected in a very sensitive and specific manner through the Dual Phage approach, as disclosed in WO 99/63348. In the Dual Phage assay two phage are needed in order to generate a signal and the two phage must be brought together or linked through the target molecule. This is most easily achieved by linking the phage to ligands such as antibodies that are specific for the target molecule. In this method it is possible to detect a wide range of target molecules including nucleic acids, proteins and simple or complex molecules.

In another approach described in U.S. Pat. No. 5,635,602 and U.S. Pat. No. 5,665,539 two target-specific antibodies are both linked to the same piece of nucleic acid, such that the nucleic acid forms a bridge. After binding to the target this nucleic acid bridge is specifically cleaved and then re-associated. The presence of an intact nucleic acid bridge (i.e. cleaved and re-associated) is shown by the use of PCR and two primers that recognise the reformed nucleic acid, because the nucleic acid bridge contains two PCR primer binding sites. This approach enhances the specificity of the assay because the nucleic acid is more likely to reform after cleavage if both antibody molecules are bound to the target and are thus in close spatial proximity. The nucleic acid bridge is unlikely to re-form if only one antibody is bound non-specifically to a molecule other than the intended target. A disadvantage of this approach is the problem of ensuring that all of the nucleic acid bridge molecules are cleaved in the absence of target antigen. In addition, the method is complex and involves a number of steps that could involve DNA restriction enzymes, DNA polymerases and DNA ligation enzymes.

WO 01/61037 relates to assays for detection of analytes in solution using so-called proximity probes. Said proximity probes consist of a binding moiety and a nucleic acid. Upon binding of the proximity probes to the analyte the nucleic acids are brought into close proximity and can thus be ligated and then detected, usually by amplification. This technique has the disadvantage that ligation can be an inefficient reaction and the ligase has to be added to the reaction in a high enough concentration to allow efficient ligation of the proximity probes.

Sensitive methods are also needed for use in monitoring molecular interactions. Drug discovery and proteomics are just two of the areas of technology that rely on the monitoring of such interactions. A variety of methods are currently in use, which are well known in the art, such as the Scintillation Proximity Assay (SPA) (Bosworth and Towers, 1989) and various Yeast Hybrid methodologies (Ma and Ptashne, 1988; Fields and Sternglanz, 1994).

The Dual Phage method can also be applied to the monitoring of molecular interactions. In this case, the molecules whose interaction is to be studied each have a ligand-binding site that can bind one phage type either directly or indirectly. The interaction of the molecules is thus able to be monitored through the linking of the two phage types. If the molecules interact the two phage types are brought together but if they do not interact the two phage types remain separate. This approach can be applied to proteomics and drug discovery.

Other approaches which can be used to detect interactions between biological molecules include fluorescent Resonance Energy Transfer (FRET), a distance-dependent excited state interaction in which emission of one fluorophore is coupled to the excitation of another. FRET relies on a fluorescence donor which transfers a quantum, or exciton, of energy to a fluorescence acceptor, thus raising an electron in the acceptor to a higher energy state as the photo-excited electron in the donor returns to the ground state. The resonance interaction between donor and acceptor fluorophores occurs over distances that are greater than interatomic. For successful FRET the fluorescent emission spectrum of the donor must overlap the absorption spectrum of the acceptor, and the donor and acceptor transition dipole orientations must be approximately parallel. The probability that energy transfer will occur depends on the sixth power of the distance between the fluorophores.

When the donor and acceptor are different, FRET can be detected by the appearance of fluorescence of the acceptor or by quenching of donor fluorescence. If the donor and acceptor are the same, FRET can be detected by the resulting fluorescent depolarization. Energy transfer can be detected by measuring emission from the acceptor fluorophore when excited at the donor fluorophore's wavelength. This wavelength does not produce an emission from the acceptor unless FRET has occurred. Alternatively, FRET can be detected by measuring the quenching effect that the acceptor has on donor emission at its excitation wavelength.

U.S. Pat. No. 3,996,345, U.S. Pat. No. 4,261,968 and U.S. Pat. No. 199,559 describe immunoassays that employ fluorescer-quencher chromophoric pairs. Here, either one or both of the chromophoric pair are bonded to antibodies. Depending on the particular ligand of interest, various reagent combinations can be employed, where the amount of quenching is directly related to the amount of ligand present in the assay medium.

U.S. Pat. No. 6,251,581 discloses methods for determining an analyte in a medium suspected of containing the analyte. One method comprises treating a medium suspected of containing an analyte under conditions such that the analyte, if present, causes a photosensitizer and a chemiluminescent compound to come into close proximity. The photosensitizer generates singlet oxygen and activates the chemiluminescent compound when in close proximity. The activated chemiluminescent compound subsequently produces light. The amount of light produced is related to the amount of analyte in the medium.

### DESCRIPTION OF THE INVENTION

The present invention seeks to provide improved methods of detecting target molecules and of monitoring molecular interactions.

In accordance with the first aspect of the invention, hereinafter referred to as the “target detection method” there is provided a method of detecting a target molecule in a sample comprising the steps of;
(a) contacting a sample with two or more binding entities specific for the target molecule, including a first binding entity labelled with a catalytic label and a second, separate binding entity labelled with a substrate label, characterised in that the catalytic label is capable of acting directly on the substrate label to generate a detectable change in the substrate label;
(b) incubating under conditions which permit the binding entities to bind to the target molecule, thus bringing the catalytic and substrate labels into close proximity;
(c) allowing the catalytic label to act directly on the substrate label when bound in close proximity, thereby producing a detectable change in the substrate label;
(d) detecting the change in the substrate label.

In accordance with a second aspect of the invention, referred to herein as the “interaction method”, there is provided a method of detecting interactions between two or more interacting molecules comprising the steps of:
(a) incubating the interacting molecules such that they can interact, thereby bringing into close proximity a catalytic label attached to one of the interacting molecules and a substrate label attached a separate interacting molecule, characterised in that the catalytic label is capable of acting directly on the substrate label to generate a detectable change in the substrate label;
(b) allowing the catalytic label to act directly on the substrate label when bound in close proximity, thereby producing a detectable change in the substrate label;
(c) detecting the change in the substrate label.

Both methods rely on specific binding interactions into order to bring into close proximity a “catalytic label” and a “substrate label”. The catalytic label is capable of acting directly on the substrate label to generate a detectable change in the substrate label. In this context “detectable change” means a change in structure and/or activity of the substrate label, caused by the action of the catalytic label, which change in structure and/or activity can be directly or indirectly detected. The “detectable change” in the substrate label is preferably irreversible under the conditions used to detect the change.

In the most preferred embodiment the “detectable change” is caused by direct action of the catalytic label on the substrate label, meaning that the change in structure and/or activity is brought about by direct contact or physical interaction between the catalytic and substrate labels. The term “direct action” excludes changes in structure and/or activity is of the substrate label which are brought about solely though the action of a diffusable intermediate, without any requirement for a direct contact or physical interaction between the catalytic and substrate labels.

The catalytic and substrate labels are attached to “separate” binding entities/interacting molecules, meaning that no part of the catalytic label is attached to the same binding entity/interacting molecule as any part of the substrate label. The catalytic and substrate labels are brought into close proximity by specific binding of binding entities to a target molecule (in the target detection method) or specific binding of interacting molecules (interaction method), thus facilitating direct action of the catalytic label on the substrate label. The close juxtaposition of the catalytic and substrate labels as a result of specific binding events results in a rate enhancement that provides an improved signal-to-noise ratio over the background due to random interactions between un-bound catalytic and substrate labels in free solution.

The target detection and interaction methods according to the invention are types of biological binding assays and may be carried out in accordance with standard principles known in the art for these types of assays. For example, it is common to include intermediate washing steps between addition of reagents to remove excess/unbound reagents, as illustrated in the accompanying examples.

Preferred Features of Catalytic and Substrate Labels

Action of the catalytic label on the substrate label results in a detectable change in structure and/or activity of the substrate label.

Changes in the structure of the substrate label may include, for example, physical cleavage of the label.

Changes in activity of the substrate label may include, for example, a change in a detectable property such as fluorescence, luminescence, etc, or a change in enzymatic activity, electrochemical activity or redox properties. The change in activity may be accompanied by, or caused by, a change in structure of the substrate label.

The “change” in activity of the substrate label may be a change in activity resulting from a discrete switch of the substrate label from an “inactive” to an “active” state (or vice versa) as a result of a discrete action of the catalytic label on the substrate label (an example being a cleavage of the substrate label). However, in other embodiments the “change” in activity may be the appearance of an activity which requires continued action of the catalytic label on the substrate label. An example of such a “change” is increased transcriptional activity resulting from interaction between an RNA polymerase catalytic label and a nucleic acid substrate label bearing a promoter specific for the RNA polymerase.

Preferred catalytic labels include, but are not limited to, enzymes.

In one embodiment of the invention the action of the catalytic label on the substrate label may generate a fluorophore, which can subsequently be detected. For example, the substrate label may be formed of a fluorescent moiety linked to a quencher moiety which quenches the fluorescence of the fluorescent moiety by via a linkage which is cleaved by the action of the catalytic label. Cleavage of the linkage releases the quencher moiety and thus increases fluorescence from the fluorescent moiety. This increase in fluorescence may be detected using a suitable measuring instrument. In this embodiment the catalytic label may be an enzyme and the “linkage” may be a molecular structure which is cleaved by the action of the enzyme. For example, the enzyme may be a protease and the linkage a peptide which is cleaved by the action of the enzyme. The precise nature of the linkage is not important, except to the extent that it must enable the fluorescent and quencher moieties to interact when “linked”, such that the quencher moiety quenches fluorescence from the fluorescent moiety.

In a further embodiment the action of the catalytic label on the substrate label may quench a fluorophore.

In a still further embodiment the action of the catalytic label on the substrate label may create an electrochemically active species or group that can subsequently be detected. For example the catalytic label may be the enzyme alkaline phosphatase and the substrate label may be a phenol phosphate moiety coupled directly to a binding moiety. In this case the enzyme, when in close proximity to the substrate, catalyses the removal of the phosphate group, generating an electrochemically active group which may be detected by a redox reaction at an electrode surface.

In a still further embodiment the substrate label may be an inactive molecule which becomes activated by the action of the catalytic label. Example of such labels include, for example, enzyme precursors, zymogens or pro-enzymes.

Many enzymes are synthesized as inactive precursors, called zymogens or pro-enzymes, which are subsequently activated by cleavage of one or a few specific peptide bonds. This proteolytic activation is an irreversible process. For example, trypsinogen, chymotrypsinogen, proelastase and procarboxypeptidase are all inactive precursors, of the digestive enzymes trypsin, chymotrypsin, elastase and carboxypeptidase, respectively. All of these pro-enzymes become activated by the action of trypsin, which hydrolyses peptide bonds in the pro-enzymes. Therefore, in a preferred embodiment of the invention the catalytic label may be trypsin, or a protease of equivalent proteolytic activity but differing specificity, and the substrate label may be one of trypsinogen, chymotrypsinogen, proelastase and procarboxypeptidase. These examples are merely illustrative and are not intended to be limiting to the invention.

Other preferred combinations of enzyme catalytic labels and enzyme precursor substrate labels include enteropeptidase/trypsinogen (cleaved to generate trypsin), thrombin/fibrinogen (cleaved to generate fibrin).

In such systems wherein the catalytic label cleaves an enzyme precursor substrate label to generate an active enzyme, the “change” brought about by action of the catalytic label on the substrate label (i.e. enzyme activation) may be conveniently detected by a suitable assay specific for the enzyme activity generated by cleavage of the substrate label. It is important that this assay is capable of distinguishing between enzymatic activity generated as a result of cleavage of the substrate label and the enzymic activity of the catalytic label.

Other preferred examples of catalytic labels which may be used in accordance with the invention include phosphatases and kinases. Such catalytic labels will, respectively, de-phosphorylate or phosphorylate a suitable substrate label. Dephosphorylation or phosphorylation of the substrate label may, in turn, lead to a detectable change in structure and/or activity of the substrate label.

In certain embodiments the substrate label which is phosphorylated/dephosphorylated by the kinase/phosphatase may be a protein, however the invention is not limited to only proteins. Any suitable substrate label which can be phosphorylated or dephosphorylated, wherein such phosphorylation or dephosphorylation is accompanied by a detectable change in structure and/or activity, may be used within the scope of the invention.

In a still further embodiment of the invention the substrate label may be a nucleic acid and the catalytic label may be a species, typically an enzyme, capable of catalysing a detectable change in the structure and/or activity of the nucleic acid. Examples of suitable catalytic labels include, for example, recombinases, ligases, transposases, DNA polymerases, reverse transcriptases or RNA polymerases.

In this embodiment, changes in the “structure” of a nucleic acid label are taken to include changes in sequence which are detectable, for example with the use of suitable sequence-specific probes and/or nucleic acid amplification techniques. There are many suitable techniques known to those skilled in the art which may be used to detect and differentiate specific nucleic acid sequences.

Changes in the “activity” of a nucleic acid label may include, as discussed previously, a change in transcriptional activity, such as may occur if the catalytic label is an RNA polymerase and the substrate label contains a promoter specific for the said polymerase. Preferred RNA polymerase/promoter systems for use in this embodiment of the invention include (but are not limited to) the bacteriophage RNA polymerases, especially T7, T3 and SP6 polymerases, and their cognate promoters. These polymerases are well known in the art and are routinely used for in vitro transcription. Increases in transcriptional activity brought about by the action of an RNA polymerase catalytic label on a substrate label containing an appropriate promoter may be detected by directly or indirectly detecting the resulting transcripts. In a preferred embodiment, the substrate label may be a reporter gene expression construct comprising a suitable promoter driving expression of a reporter gene. The increase in transcriptional activity may then be monitored by detecting expression of the reporter gene.

In one embodiment of the invention, the substrate label may be formed of two or more component parts, at least one of which is attached to a separate binding entity/interacting molecule to the catalytic label. There are various ways in which this can be achieved. For example, the component parts of the substrate label may be all attached to a single binding entity/interacting molecule and the catalytic label may be attached to a separate binding entity/interacting molecule.

In a further arrangement, the catalytic label and component parts of the substrate label may each be attached to separate binding entities/interacting molecules. Such a system has the advantage that three species must be brought into close proximity for the interaction between catalytic and substrate labels to take place. Thus, in a “target detection method” wherein the catalytic label and components of the substrate label are each attached to separate binding entities, at least three separate binding events of binding entities to a common target must take place in order to enable the interaction between catalytic and substrate labels. In the “interaction method”, wherein at least three separate interacting molecules are respectively labelled with a catalytic label and components of the substrate label, then at least three separate species of interacting molecules must come together, in order to enable the interaction between catalytic and substrate labels.

In a still further embodiment, the catalytic label and at least one component part of the substrate label may each be attached to separate binding entities/interacting molecules and a further component part of the substrate label may be present in free solution, preferably in an excess amount. In this system there will still be a “proximity effect” between the catalytic label and the component of the substrate label which is attached to a binding entity/interacting molecule, as these two labels will only be brought into proximity by binding of labelled binding entities to their target(s) or interaction of labelled interacting molecules.

An example of a substrate label formed of two component parts is a substrate label formed of two separate nucleic acid tags, which are capable of interacting, in the presence of a suitable catalytic label, to generate at least one nucleic acid tag having novel sequence. This type of label is particularly useful in an assay system wherein one nucleic acid tag is attached to a binding entity/interacting molecule and another nucleic acid tag is present in free solution in excess.

In the most preferred embodiment of the system wherein substrate label formed of two separate nucleic acid tags, the tags are capable of interacting via recombination to generate at least one tag of novel sequence. In this embodiment the catalytic label comprises an enzyme that catalyses recombination between the nucleic acid tags.

“Recombination” is defined herein to include any exchange of nucleic acid sequence or deletion or insertion of sequences between the nucleic acid tags in order to generate at least one novel sequence that is capable of being detected. Examples include site-specific recombination events (e.g. requiring a specific recombinase) and transposition events (e.g. requiring a specific transposase).

Site-specific recombination events are non-homologous recombination events, in so far as they generally do not require extensive homology between nucleic acid tags. In most cases site-specific recombination requires the presence of short recombination site sequences (generally a few tens of base-pairs). Many site-specific recombination systems require the presence of identical recombination site sequences. However, in other systems the recombination sites may share little or no sequence homology, as is the case with the integration sites attP and attB, derived respectively from bacteriophage lambda and the E. coli chromosome.

In a preferred embodiment, the “substrate label” is comprised of two nucleic acid tags, each containing a site-specific recombination sequence recognised by a particular site-specific recombinase enzyme, and the “catalytic label” is the appropriate site-specific recombinase enzyme.

Suitable site-specific recombination systems which may be used include the Cre/loxP system, wherein the nucleic acid tags making up the substrate label contain loxP sites, and the catalytic label is Cre recombinase. Another suitable system is the bacteriophage lambda integration system, wherein the nucleic acid tags contain attP and attB recognition sequences or attL and attR sequences, allowing recombination catalysed by an enzyme label which recognises these sites. Recombination between attB and attP sites or between attL and attR sequences is catalysed by the lambda phage enzyme integrase, and requires a host accessory factor IHF. The lambda phage recombination system is well known in the art and the enzymes required for recombination are available commercially (e.g. as components of the Gateway™ cloning system supplied by Invitrogen). These particular recombination systems are listed by way of example only and it is not intended to limit the invention to the use of these specific systems. Other site-specific recombination systems known in the art such as, for example the Flp/FRT system, may also be used.

In a particular embodiment of the “recombinase” system, the recombinase enzyme may actually be attached to one of the nucleic acid tags making up the substrate label. For example, the two nucleic acid tags making up the recombinase substrate may be attached to separate binding entities/interacting molecules and the recombinase enzyme may be attached to one of the nucleic acid tags. Upon binding of the binding entities to their target/interaction between the interacting molecules the two nucleic acid tags plus the recombinase will be brought into close proximity, thus enabling the recombination reaction to take place. For the majority of applications it will, however, be preferable to have the recombinase and nucleic acid tags attached to separate components.

In a still further embodiment, recombination may depend upon a transposition event and rely upon the use of a transposase as the catalytic label. A suitable example of such a system depends upon Tn5 transposase that recognizes Mosaic Ends recognition sequences. However, it is not intended to limit the invention to the use of this specific system, and other transposition systems known in the art may be used.

Systems which rely on the use of a transposase as the catalytic label in order to generate a “detectable change” by transposition of a nucleic acid sequence between two nucleic acid tags making up the substrate label may or may not require the presence of specific sequences in both the nucleic acid tags in order to allow transposition to take place. The requirements for successful transposition with any particular transposase enzyme/transposable element will generally be appreciated by those skilled in the art.

A further preferred embodiment of a system wherein the substrate label is composed of two nucleic acid tags is based on the use of a ligase as the catalytic label. The ligase is capable of ligating together two separate nucleic acid tags, which together make up the substrate label, in order to form a detectable ligation product having novel sequence. In this system it is possible to attach one of the nucleic acids to a binding entity/interacting molecule and add the second tag in free solution in an excess amount.

The nucleic acid tags used in this embodiment may be formed of double-stranded RNA, enabling ligation by T4 DNA ligase. The tags may have complementary “sticky” ends or blunt ends. In other embodiments, the tags may be formed of single-stranded RNA, which can be joined by T4 RNA ligase.

Nucleic acid tags having novel sequence, such as may be generated by the action of recombinases, transposases or ligases on “substrate” nucleic acid tags may be detected using any suitable technique known in the art.

Most preferably, detection of the novel sequence will involve an amplification reaction, for example PCR, NASBA, 3SR or any other amplification technique known in the art. Amplification is achieved with the use of amplification primers specific for the novel sequence. In order to provide specificity for the novel tag sequence primer binding sites corresponding to a region of completely novel sequence may be selected, or else a novel combination of primer binding sites, not present in the original tags, may be chosen.

The skilled reader will appreciate that the novel sequence may also include sequences other than primer binding sites which are required for detection of the novel sequence, for example RNA Polymerase binding sites or promoter sequences required for isothermal amplification technologies, such as NASBA or 3SR.

In a preferred embodiment detection of the novel sequence is carried out by amplification with “real-time” detection of the products of the amplification reaction. This can be achieved using any amplification technique which allows for continuous monitoring of the formation of the amplification product.

A number of techniques for real-time detection of the products of an amplification reaction are known in the art. Many of these produce a fluorescent read-out that can be continuously monitored, specific examples being molecular beacons and fluorescent resonance energy transfer probes. Real-time quantification of PCR reactions can be accomplished using the TaqMan® system (Applied Biosystems).

In a most preferred embodiment the method is carried out in real-time, meaning that specific binding of the binding entities to the target molecule (or specific binding of interacting molecules), action of the catalytic label on the nucleic acid substrate label and detection of the product of the interaction are carried out simultaneously in a single reaction step. Real-time detection requires that the binding step, action of the catalytic label on the substrate label, and detection of the product of the interaction can all be carried out under a single set of reaction conditions, without the need for intermediate washing steps. In this embodiment real-time detection of the novel sequence will preferably be carried out using an isothermal amplification reaction, for example NASBA or 3SR, in order to avoid changes of temperature which might adversely affect the binding of the binding entities to the target molecule/interaction between interacting molecules.

In embodiments wherein the substrate label is comprised of nucleic acid tags, the term “nucleic acid tags” includes any natural nucleic acid and natural or synthetic analogues that can be acted upon by a catalytic label to generate novel sequence, for example by recombination. Suitable nucleic acid tags include tags composed of double or single-stranded DNA, double or single-stranded RNA. Tags which are partially double-stranded and partially single-stranded are also contemplated. It is also contemplated to use single-stranded tags in combination with double-stranded tags, i.e. one component labelled with a single-stranded tag and another component labelled with a double-stranded tag capable of interacting with the single-stranded tag. If the catalytic label catalyses a recombination, then the nucleic acid tags may be composed of any nucleic acid which is capable of participating in the recombination reaction, suitable examples including linear or circular double-stranded DNA (dsDNA) or double-stranded RNA (dsRNA) or mixtures thereof. Most preferably the nucleic acid tags will comprise dsDNA. The term “nucleic acid” encompasses synthetic analogues which form a substrate for the catalytic label in an analogous manner to natural nucleic acids, for example nucleic acid analogues incorporating non-natural or derivatized bases, or nucleic acid analogues having a modified backbone. In particular, the term “double-stranded DNA” or “dsDNA” is to be interpreted as encompassing dsDNA containing non-natural bases.

The precise sequence of the nucleic acid tags is not material to the invention, except to the extent that certain sequences may be required to enable the “action” of the catalytic label on the nucleic acid tags. For example, specific sequences are required to permit site-specific recombination. Two nucleic acid tags making up a “substrate label” will most usually be of different sequence, so that an interaction event between the nucleic acid tags leads to production of at least one novel sequence that can be detected. However, it is not excluded to use tags of identical sequence, provided that the tags are able to interact to generate novel sequence. Most preferably, the action of the catalytic label on the substrate label will lead to the production of two separate nucleic acid tags, each having novel sequence. This has the advantage that the two tags of novel sequence form independently verifiable products.

In a further embodiment of the invention, the catalytic label may be formed of two or more component parts, at least one of which is attached to a separate binding entity/interacting molecule to the substrate label. Examples of such catalytic labels may include, for example enzyme/co-enzymes, multi subunit enzymes, etc.

The substrate label and component parts of the catalytic label may each be attached to separate binding entities/interacting molecules, or the component parts of the catalytic label may all be attached to a single binding entity/interacting molecule and the substrate label may be attached to a separate binding entity/interacting molecule.

In a still further embodiment, the substrate label and catalytic label may each be formed of two or more component parts, wherein component parts of the substrate and catalytic labels are attached to separate binding entities/interacting molecules. In this embodiment all component parts of the substrate label may be attached to a single binding entity/interacting molecule or the component parts may be attached to a number of separate binding entities/interacting molecules. Similarly, all component parts of the catalytic label may be attached to a single binding entity/interacting molecule or the component parts may be attached to a number of separate binding entities/interacting molecules. The only limitation is that (component parts of) the substrate and catalytic labels must not be attached to the same binding entity/interacting molecule.

It is generally preferred to start with the catalytic label in an inactivated state and then activate the catalytic label only after binding of the binding entities to the target molecule (or interaction between interacting molecules) has taken place to position the catalytic and substrate labels in close proximity. Activation of the catalytic label may, for example, be achieved by changing the composition, pH or temperature of the reaction medium. This “external activation” step increases the sensitivity of the method by minimising background resulting from non-specific interactions between the catalytic label and substrate label in free solution. This may be important in applications wherein the “proximity effect” which results from specific binding of binding entities to their target molecule or specific interactions between interacting molecules in order to bring the catalytic and substrate labels into close proximity does not by itself provide sufficient signal-to-noise enhancement over background non-specific interactions.

In a particular embodiment, step (b) of the target detection method and the equivalent step (a) of the interaction method may be carried out under conditions which do not permit the catalytic label to act on the substrate label. These “conditions” may be, for example, the lack of a key component required for the action of the catalytic label on the substrate label. If the substrate label is formed of two component parts, one of which is to be added in free solution, then the “conditions” could be the absence of this component. After binding of the binding entities to the target molecule (or interaction between interacting molecules) has taken place to position the catalytic and substrate labels in close proximity, the “conditions” may be changed to permit the catalytic label to act on the substrate label. This change could be, for example, addition of the missing component of the substrate label under conditions which permit subsequent reaction between the catalytic label and components of the substrate label. A “change” in reaction medium may easily be achieved with the use of an intermediate washing step or by simple addition of a missing reaction component (both of which are in accordance with standard principles of biological binding assays).

In embodiments wherein the substrate label is formed of two (or more) separate nucleic acid tags which are to be brought together in an interaction requiring hybridisation between the two tags, it may be convenient to carry out step (b) of the target detection method and the equivalent step (a) of the interaction method under conditions which do not allow hybridisation between the tags and then to change the reaction conditions to permit hybridisation.

In a preferred embodiment the catalytic label and substrate label are directly attached to the binding entities or interacting molecules. Direct linkage may be achieved via a covalent linkage.

Techniques are known in the art for direct covalent linkage of protein components to other proteins, or to nucleic acids or carbohydrates, see for example the techniques described in manuals such as Bioconjugation; M Aslam and A Dent, eds. Macmillan Reference Ltd 1998.

For example, amine-derivatized nucleic acid tags may be coupled to protein binding entities/interacting molecules using any one of a number of chemical cross-linking compounds.

It is also within the scope of the invention for the catalytic or substrate labels to be attached indirectly to the binding entities/interacting molecules. For example, “indirect” attachment may be achieved through linker molecules. Suitable linker molecules include components of biological binding pairs which bind with high affinity, for example biotin/streptavidin or biotin/avidin.

For most applications of the target detection method, the catalytic and substrate labels will be attached to the binding entities/interacting molecules at the start of the reaction, at least before the binding of the binding entities to the target molecule. Most preferably, the binding entities will be supplied pre-labelled with catalytic and substrate labels, or else the labels will be attached in a separate reagent labelling step. However, the possibility of attaching catalytic and/or substrate labels to the binding entities during the detection reaction itself, i.e. following binding of the binding entities to the target, is not excluded.

Preferred Features of Target Detection Method

The “target detection method” may be used to detect essentially any target molecule for which it is desired to develop a specific target detection method.

The target molecule may comprise a single molecule, a multimer, aggregate or molecular collection or complex. A multimer will generally comprise a number of repeats of a single molecule linked together through covalent or non-covalent interactions. A complex will generally consist of different molecules interacting through covalent or non-covalent interactions.

In the context of this invention “binding entities” are defined as any molecule that can bind specifically to a target molecule. Binding entities include, for example, antibodies, lectins, receptors, transcription factors, cofactors and nucleic acids, and fragments thereof which retain target-specific binding activity (e.g. Fab fragments). This list is merely illustrative and is not intended to be limiting to the invention.

The binding entities may bind different regions of a single target molecule. Thus, the catalytic and substrate labels will be brought into close proximity when the binding entities bind to their respective regions of the target molecule.

If the target molecule is a multimer or aggregate, then the binding entities may bind to equivalent binding sites on the monomeric components of the multimer or units making up the aggregate.

The “target detection method” of the invention may be adapted for the detection of essentially any “target molecule” for which suitable “binding entities” of the required specificity are available. The “sample” to be tested using the method may be essentially any material which permits the specific binding reactions that are essential to the operation of the target detection method.

The “target detection method” is of use in all areas of technology where it is desirable to provide specific detection of target molecules, in particular target biological molecules such as proteins, nucleic acids, carbohydrates, etc. One important area of application of the target detection method, though not intended to be limiting, is in the field of clinical diagnostics. Typically the “sample” will be a sample of biological fluid, e.g. whole blood, serum, plasma, urine etc, taken from a human patient. Other important applications may include the field of environmental testing and monitoring.

Preferred Features of Interaction Method

The “interaction method” may be used in essentially any field of technology where it is desired to monitor interactions between molecules, and particularly interactions between biological molecules.

In a preferred embodiment, the interaction method may be used in proteomics in order to investigate molecular interactions. For example a first interacting molecule may be labeled with either the catalytic or the substrate label, and a library of molecules which may potentially interact with the first interacting molecule may then each be labeled with the other label type. If an interaction occurs between the first interacting molecule and a component from the library of molecules, this brings the catalytic and substrate labels into close proximity, thus allowing interaction to generate a change in structure and/or activity of the substrate label, which can be detected in order to identify interacting partners.

A further application is in the field of drug discovery. For example, the interaction method may be used to study interactions between particular combinations of molecules and to identify potential inhibitors or enhancers of molecular interactions. Potential inhibitors of a given interaction could be identified by screening for the ability to reduce the signal detected following interaction of catalytic and substrate labels brought into close proximity by interaction between the interacting molecules.

The “interacting molecules” may be essentially any combination of interacting molecules which it is desired to study. These may be, for example, subunits of a multi-subunit complex, a pair of monomers making up a dimer, a ligand and receptor, an enzyme and substrate or inhibitor, etc.

The “interaction method” differs from the target detection method only in that the catalytic and substrate labels are attached to the interacting molecules which it is desired to evaluate, rather than to binding entities capable of binding to a target molecule. The interaction method may therefore incorporate analogous features to those described above in connection with the target detection method, as would be apparent to the skilled reader.

It is particularly preferred to carry out the interaction method in real-time. The ability to monitor molecular interactions in real-time provides significant advantages, particularly in the field of drug discovery.

Reagent Kits and Reagent Labelling Kits

The invention also relates to reagent kits suitable for use in carrying out the target detection method or the interaction method of the invention.

Reagent kits suitable for use in carrying out the “target detection method” may comprise a first binding entity labelled with a catalytic label and a second binding entity labelled with a substrate label, characterised in that the catalytic label is capable of acting directly on the substrate label to generate a detectable change in the substrate label.

Reagent kits suitable for use in carrying out the “interaction method” may comprise a first interacting molecule labelled with a catalytic label and a second interacting molecule labelled with a substrate label, characterised in that the catalytic label is capable of acting directly on the substrate label to generate a detectable change in the substrate label.

The reagent kits may incorporate any of the preferred features mentioned in connection with the target detection and interaction methods. Preferred combinations of catalytic and substrate labels are as listed above in the description of the target detection and interaction methods.

Reagent kits may further include supplies of suitable reaction buffer(s) and also reagents required for detection of the “detectable change” brought about by action of the catalytic label on the substrate label. For example, where the “change” in activity of the substrate label is a change in enzymatic activity, the kit may include assay reagents for use in measuring this enzymatic activity. Where the “change” is generation of a nucleic acid tag of novel sequence, such as may be detected by nucleic acid amplification, the kit may also include reagents required for the amplification reaction, for example: primer sets, amplification enzymes, probes for detection of the amplification product (including probes labelled with fluorescent or other revealing labels), positive control amplification templates, reaction buffers etc.

The invention still further provides a reagent labelling kit comprising a catalytic label and a substrate label, characterised in that the catalytic label is capable of acting directly on the substrate label to generate a detectable change in the substrate label, and means for attaching the catalytic label and substrate label to interacting molecules or to binding entities.

In one embodiment the means for attaching the tags to interacting molecules or binding entities may be a chemical reagent capable of cross-linking the catalytic label and/or the substrate label to a binding entity or interacting molecule.

In a further embodiment the “means for attaching the tags” may be an indirect linkage. Preferred types of indirect linkage are provided by components of a biological binding pair, for example biotin/avidin or biotin/streptavidin. In this embodiment the catalytic label and/or substrate label is conjugated with one half of the biological binding pair, enabling linkage to a binding entity or interacting molecule conjugated to the other half of the biological binding pair. The kit may contain a supply of pre-conjugated catalytic and substrate labels, or may include tags which have not yet been conjugated together with means for conjugating the catalytic or substrate labels with half of the binding pair. The kit will further include either binding entity or interacting molecule pre-conjugated with the other half of the binding pair, or else means for conjugating a binding entity or interacting molecule of choice to the other half of the binding pair.

The means for attaching half of the biological binding pair to a binding entity or interacting molecule may (depending on the nature of the binding pair) be a chemical cross-linking reagent. However, it may comprise an expression vector which can be used to express the binding entity or interacting protein as a fusion protein, either as a direct fusion with the other half of the binding pair or as a fusion with a polypeptide tag which enables attachment of the other half of the binding pair. By way of example, vectors for the expression of biotinylated fusion proteins are known in the art and are commercially available (for example the PinPoint vector system from Promega, Madison, Wis., USA). These vectors allow expression of proteins as fusions with a biotinylation domain of the biotin carboxylase carrier protein. The fusion proteins can be biotinylated in E. coli host cells in an ATP-dependent enzymic reaction. Thus, the reagent labelling kit may contain a supply of such a vector, which enables expression of biotinylated binding entities/interacting molecules proteins, plus streptavidin conjugated catalytic and/or substrate labels.

The invention will be further understood with reference to the following non-limiting experimental examples, together with the accompanying drawings, in which:

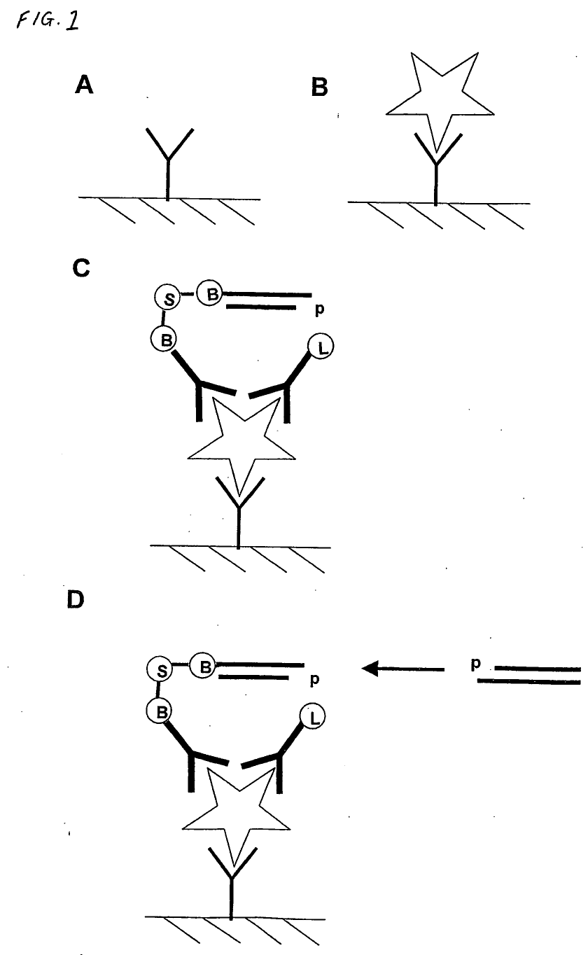

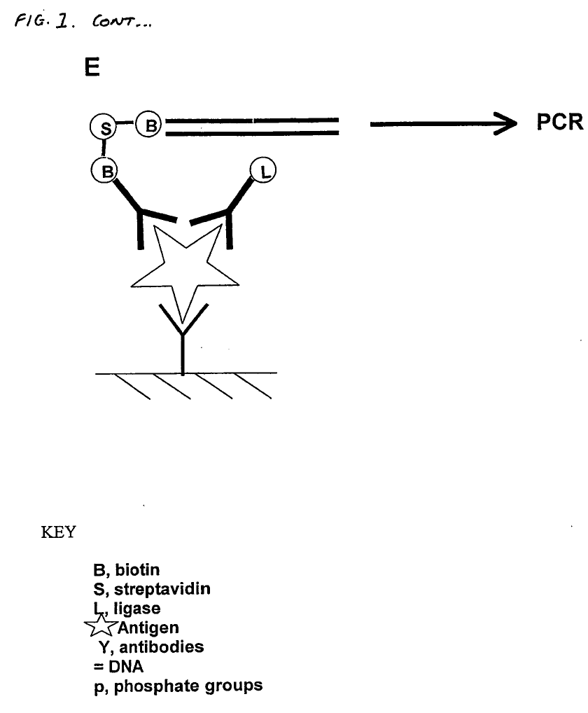

FIG. 1 is a schematic illustration of a “target detection” assay according to the invention.

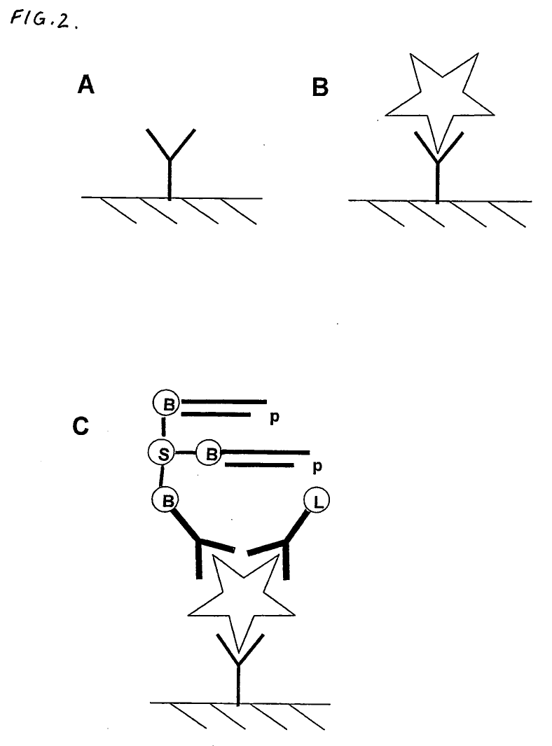

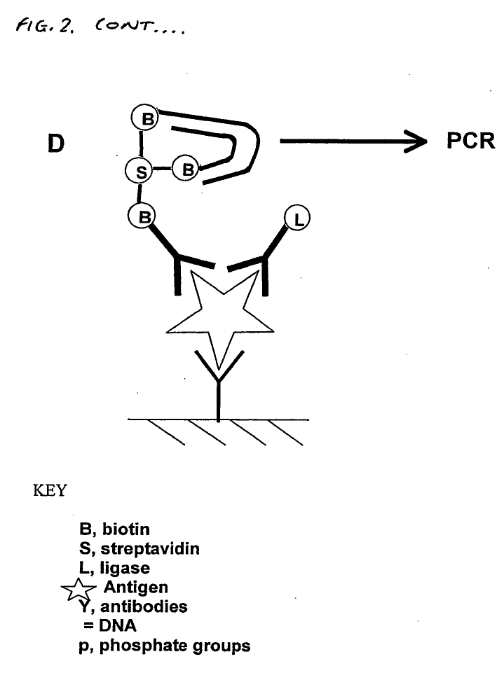

FIG. 2 is a schematic illustration of a further “target detection” assay according to the invention.

### EXAMPLE 1

### Demonstration of Detectable DNA Modification by Close Proximity Approach of a Binding Entity Modified with a DNA Substrate and a Binding Entity Modified with an Enzyme

This experiment illustrates that DNA substrates on one binding entity can be modified in a detectable manner by an DNA-specific enzyme on another binding entity under conditions such that the binding entitles are brought into close proximity by binding to a target molecule (see FIG. 1). In this example a double stranded piece of DNA with a single-stranded overhang is linked to a polyclonal anti-hepatitis C core antibody through a biotin-streptavidin bridge. Another antibody with specificity for the core is labelled with T4 DNA ligase. In the assay the target antigen, Hepatitis C virus core protein, is first immunocaptured to a solid phase surface and then incubated with the labelled antibodies under conditions which allow antibody binding but which do not allow the enzyme on one antibody to modify the DNA on the other. After binding of the antibody conjugates and removal of excess conjugate by washing a DNA oligomer with a complementary single-strand sequence to that in the antibody/DNA conjugate is added in buffer conditions that allow hybridization. If the two antibodies labelled with the DNA and the ligase respectively are in close proximity then the hybridised DNA strands can be ligated to form a stable double stranded DNA molecule which can then be detected by nucleic acid amplification methods, such as PCR.

a) Synthesis of the DNA-Anti-Hepatitis C Core Antibody Conjugate

1. Two overlapping oligonucleotides were constructed:

322S1;
5′CGGGCCTCTT GCGGGATATC GTCCATTCCG ACAGCATCGC
CAGTCACTAT GGCG3′
322AS1;
5′ATAGTGACTG GCGATGCTGT CGGAATGGAC GATATCCCGC
AAGAGGCCCG3′

322S1 was synthesized with a biotin at the 5′end to allow conjugation to streptavidin whereas 322AS1 was synthesized with a phosphate at the 5′ end to allow subsequent ligation.

2. To form double-stranded DNA with a 4 bp single-stranded phosphate terminated sequence extension the oligos were mixed at a 50 pmol/μl concentration in 10 mM Tris pH 8.0, 1 mM EDTA, 150 mM NaCl and incubated at 37° C. for 1 hour.

3. The double-stranded DNA product was complexed with streptavidin to form a conjugate with a 1:1 streptavidin/DNA molar ratio. The reaction was incubated at room temperature (22° C.) for 1 hour to allow the complex formation.

4. This double-stranded DNA-streptavidin complex was further complexed to anti-hepatitis C core antibody by adding biotinylated antibody (biotinylated according to standard biotinylation protocols) at a 1:1 streptavidin/antibody molar ratio. The reaction was incubated at room temperature (22° C.) for 1 hour to allow the final conjugate formation. In this example the conjugate was then used in the assay procedure without prior removal of any free streptavidin or antibody.

b) Synthesis of the Ligase-Anti-Hepatitis C Core Antibody Conjugate

1. T4 DNA ligase was obtained from Sigma (Poole, UK) and linked to the anti-hepatitis C core antibody using the heterobifunctional reagent SMCC (Pierce Co) following the supplier's recommended standard chemical conjugation procedures. A molar ration of 2:1 ligase to antibody was used in the coupling procedure. In this example the conjugate was used in the assay procedure without prior removal of any free ligase or antibody.

c) Synthesis of the Double-Stranded DNA Used in Step 6 Below.

1. Two overlapping oligonucleotides were constructed:

| | |
|---|---|
| i. 322S2; | TGCTGCTAGC GCTATATGCG TTGATGCAAT |
| | TTCTATGCGC ACCCGTTCTC GGAGCA3′ |
| ii. 322AS2; | 5′TGCTCCGAGA ACGGGTGCGC ATAGAAATTG |
| CATCAACGCA TATAGCGCTA GCAGCACGCC3′ | |

322S2 was synthesized with a phosphate at the 5′ end to allow subsequent ligation.

2. To form double-stranded DNA with a 4 bp single-stranded phosphate terminated sequence extension the oligos were mixed at a 50 pmol/μl concentration in 10 mM Tris pH 8.0, 1 mM EDTA, 150 mM NaCl and incubated at 37° C. for 1 hour.

d) Detection of HCV Recombinant Core Antigen

1. Microwells in a 96 well microplate were coated with anti-hepatitis C core antibody at 10 μg/ml in 50 mM carbonate buffer pH 9.0 (see A in FIG. 1).

2. Serial dilutions of recombinant core antigen were prepared in PBS buffer containing 0.1% (v/v) Tween 20, 1 mM EDTA. 100 μl of each dilution was incubated in an antibody coated microplate well for 60 min to allow capture (see B in FIG. 1).

3. Wells were washed ×3 with PBS buffer pH 7.5, 0.1% (v/v) Tween 20, 1 mM EDTA.

4. 10 ng of each antibody conjugate in 100 μl 10 mM sodium phosphate buffer, 0.1% Tween 20, 100 mM NaCl, 1 mM EDTA pH 7.5 was added and incubated for 30 min to allow the conjugates to bind to the captured antigen (see C in FIG. 1).

5. Wells were washed ×3 in the same buffer and then ×2 in buffer without EDTA and Tween 20.

6. Then 100 μl 10 mM sodium phosphate buffer pH 7.5, 100 mM NaCl, 1 mM ATP, 10 mM MgCl₂ pH 7.5 containing 10 ng double-stranded DNA with a 4 bp complementary overhang to that of the conjugate DNA was added and incubated 60 min at 37° C. to allow hybridization and ligation to take place (see D and E in FIG. 1).

7. After ligation, the generation of newly covalently-linked double stranded DNA was investigated using a ‘hot-start’ quantitative PCR on a Light Cycler using standard conditions. The primers: 322PCRAS 5′ACGGGTGCGC ATAGAAATTG CATC3′ and 322PCRS 5′GCGGGATATC GTCCATTCCG ACAG3′ were used, which amplify across the junction at which the two DNA fragments have been ligated. The amount of DNA amplified in the PCR and the time/cycle of PCR at which the PCR becomes positive is related to the number of ligated DNA fragments present in the reaction, which in turn is related to the number of antibody conjugates brought into close proximity and is therefore a measure of the number of HCV core antigen molecules captured.

e) Results

The time and number of cycles at which the PCR became positive was related to the amount of antigen captured in the well. As little as 0.1 fg of HCV core antigen target could be detected by this method.

f) Discussion

This experiment shows that antibody conjugates labelled with DNA substrate and a DNA-dependent enzyme that modifies the DNA in a detectable manner can be used to detect antigen in a sensitive and specific manner.

### EXAMPLE 2

### Demonstration of Detectable DNA Modification by Close Proximity Binding of a Binding Entity Modified with Two DNA Substrates and a Binding Entity Modified with an Enzyme

This experiment illustrates that two DNA substrates on one binding entity can be modified in a detectable manner by a DNA-specific enzyme on another binding entity under such conditions such that the binding entities are brought into close proximity by binding to a target molecule (see FIG. 2). In this example two double stranded DNA oligomers with single-stranded sequence extensions are linked to a target-specific antibody (a polyclonal anti-hepatitis C core antibody) through a biotin-streptavidin bridge. Another target specific antibody is labelled with T4 DNA ligase. In the assay the target antigen is immunocaptured to a solid phase and then incubated with the labelled antibodies under conditions which allow antibody binding but do not allow the enzyme on one antibody to modify the DNA on the other. After binding of the antibody conjugates and removal of excess conjugate by washing, the buffer conditions are then changed to allow hybridisation. If the two antibody conjugates (conjugated to the two DNA oligomers and the ligase respectively) are in close proximity the two DNA strands can be ligated to yield a stable double-stranded DNA product which can then be detected by nucleic acid amplification methods such as PCR.

a) Formation of the DNA-Anti-Hepatitis C Core Antibody Conjugate

1. Two overlapping oligonucleotides were constructed:

322S1;
5′CGGGCCTCTT GCGGGATATC GTCCATTCCG ACAGCATCGC
CAGTCACTAT GGCG3′
322AS1;
5′ATAGTGACTG GCGATGCTGT CGGAATGGAC GATATCCCGC
AAGAGGCCCG3′

322S1 was synthesized with a biotin at the 5′end to allow conjugation to streptavidin whereas 322AS1 was synthesized with a phosphate at the 5′ end to allow subsequent ligation.

2. To form double-stranded DNA with a 4 bp single-stranded phosphate terminated sequence extension the oligos were mixed at a 50 pmol/μl concentration in 10 mM Tris pH 8.0, 1 mM EDTA, 150 mM NaCl and incubated at 37° C. for 1 hour.

3. Two further overlapping oligonucleotides were constructed:

322S2;
TGCTGCTAGC GCTATATGCG TTGATGCAAT TTCTATGCGC
ACCCGTTCTC GGAGCA3′
322AS2;
5′TGCTCCGAGA ACGGGTGCGC ATAGAAATTG CATCAACGCA
TATAGCGCTA GCAGCACGCC3′

322S2 was synthesized with a phosphate at the 5′ end to allow subsequent ligation.

4. To form double-stranded DNA with a 4 bp single-stranded phosphate terminated sequence extension the oligos were mixed at a 50 pmol/μl concentration in 10 mM Tris pH 8.0, 1 mM EDTA, 150 mM NaCl and incubated at 37° C. for 1 hour.

5. These double-stranded DNA products were linked to streptavidin by mixing the two DNAs and the streptavidin at equimolar concentration. The reaction was incubated at room temperature for 1 hour to allow the complex formation to take place.

6. This double-stranded DNA-streptavidin complex was complexed to anti-hepatitis C core antibody by adding biotinylated antibody (biotinylated according to standard biotinylation protocols) at a 1:1 molar ratio relative to the streptavidin. The reaction was incubated at room temperature for 1 hour to allow the complex formation to take place. In this example the conjugate was used in the assay without removal of any free streptavidin or antibody.

b) Formation of the Ligase-Anti-Hepatitis C Core Antibody Conjugate

1. T4 DNA ligase was obtained from Sigma (Poole, UK) and linked to the anti-hepatitis C core antibody using the heterobifunctional reagent SMCC (Pierce Co) following the supplier's recommended standard chemical conjugation procedures. A molar ration of 2:1 ligase to antibody was used in the coupling procedure. In this example the conjugate was used in the assay procedure without prior removal of any free ligase or antibody.

c) Detection of HCV Recombinant Core Antigen

1. Microwells in a 96 well microplate were coated with anti-hepatitis C core antibody at 10 μg/ml in 50 mM carbonate buffer pH 9.0 (see A in FIG. 2). Serial dilutions of recombinant core antigen were made in PBS buffer pH 7.2, 0.1% (v/v) Tween 20, 1 mM EDTA and 100 μl of each dilution incubated in a coated well for 60 min to allow capture (see B in FIG. 2).

2. Wells were washed ×3 with PBS buffer pH 7.2 0.1% (v/v) Tween 20, 1 mM EDTA.

3. 10 ng of each antibody conjugate in 100 μl 10 mM sodium phosphate buffer, 0.1% Tween20, 100 mM NaCl, 1 mM EDTA pH 7.5 was added and incubated for 30 min to allow the antibody conjugates to bind to the antigen (see C in FIG. 2).

4. Wells were washed ×3 in the same buffer and then ×2 in buffer without EDTA and Tween 20.

5. Then 100 μl 10 mM sodium phosphate, 100 mM NaCl, 1 mM ATP, 10 mM MgCl₂ pH 7.5 was added and incubated 60 min at 37° C. to allow hybridization, ligation and the formation of covalently linked DNA fragments (see D in FIG. 2).

6. After ligation, the generation of covalently-linked double stranded DNA was investigated using a ‘hot-start’ quantitative PCR on a Light Cycler using standard conditions. The primers: 322PCRAS 5′ACGGGTGCGC ATAGAAATTG CATC3′ and 322PCRS 5′GCGGGATATC GTCCATTCCG ACAG3′ were used which amplify across the junction at which the two DNA fragments join. The amount of DNA amplified in the PCR and the time/cycle of PCR at which the reaction becomes positive is related to the number of ligated DNA fragments present in the reaction, which in turn is related to the number of antibody conjugates brought into close proximity and is therefore a measure of the number of HCV core antigen molecules captured.

d) Results

The time and number of cycles at which the PCR became positive was related to the amount of antigen captured in the well. As little as 0.2 fg of antigen target could be detected by this method.

e) Discussion

This experiment shows that antibodies labelled, respectively, with two DNA substrates and a DNA-dependent enzyme that modifies the DNA in a detectable process can be used to detect antigen in a sensitive and specific manner.

1. A method of detecting a target molecule in a sample comprising the steps of;
(a) contacting a sample with two or more binding entities specific for the target molecule, including a first binding entity labelled with a catalytic label and a second, separate binding entity labelled with a substrate label, characterised in that the catalytic label is capable of acting directly on the substrate label to generate a detectable change in the substrate label;
(b) incubating under conditions which permit the binding entities to bind to the target molecule, thus bringing the catalytic and substrate labels into close proximity;
(c) allowing the catalytic label to act directly on the substrate label when bound in close proximity, thereby producing a detectable change in the substrate label;
(d) detecting the change in the substrate label.

2. A method of detecting interactions between two or more interacting molecules comprising the steps of:
(a) incubating the interacting molecules such that they can interact, thereby bringing into close proximity a catalytic label attached to one of the interacting molecules and a substrate label attached to a separate interacting molecule, characterised in that the catalytic label is capable of acting directly on the substrate label to generate a detectable change in the substrate label;
(b) allowing the catalytic label to act directly on the substrate label when bound in close proximity, thereby producing a detectable change in the substrate label;
(c) detecting the change in the substrate label.

3. A method according to claim 1 or claim 2 wherein the detectable change is a change in the structure and/or activity of the substrate label.

4. A method according to any one of claims 1 to 3 wherein the detectable change is irreversible.

5. A method according to any one of claims 1 to 4 wherein the catalytic label and substrate label are directly attached to the binding entities or interacting molecules.

6. A method according to any one of the preceding claims wherein the substrate label is formed of two or more component parts, at least one of which is attached to a separate binding entity/interacting molecule to the catalytic label.

7. A method according to claim 6 wherein the catalytic label and component parts of the substrate label are each attached to separate binding entities/interacting molecules.

8. A method according to claim 6 wherein the component parts of the substrate label are all attached to a single binding entity/interacting molecule and the catalytic label is attached to a separate binding entity/interacting molecule.

9. A method according to claim 6 wherein a component part of the substrate label is added in free solution.

10. A method according to any one of claims 1 to 5 wherein the catalytic label is formed of two or more component parts, at least one of which is attached to a separate binding entity/interacting molecule to the substrate label.

11. A method according to claim 10 wherein the substrate label and component parts of the catalytic label are each attached to separate binding entities/interacting molecules.

12. A method according to claim 10 wherein the component parts of the catalytic label are all attached to a single binding entity/interacting molecule and the substrate label is attached to a separate binding entity/interacting molecule.

13. A method according to any one of claims 1 to 5 wherein the substrate label and catalytic label are each formed of two or more component parts, wherein the component parts of each of the substrate and catalytic labels are attached to separate binding entities/interacting molecules.

14. A method according to any of the preceding claims wherein the catalytic label, or a component part thereof, is an enzyme.

15. A method according to any of claims 1 to 14 wherein the action of the catalytic label on the substrate label generates a fluorophore.

16. A method according to any of claims 1 to 14 wherein the action of the catalytic label on the substrate label quenches a fluorophore.

17. A method according to any of claims 1 to 14 wherein the action of the catalytic label on the substrate label creates an electrochemically active group that can be subsequently detected.

18. A method according to any of claims 1 to 14 wherein the substrate label is an inactive molecule which becomes activated by the action of the catalytic label.

19. A method according to claim 18 wherein the substrate label is an enzyme precursor, pro-enzyme or zymogen.

20. A method according to claim 18 or claim 19 wherein the catalytic label, or a component part thereof, is a protease.

21. A method according to any of claims 1 to 14 wherein the substrate label, or the component parts thereof, is/are nucleic acid and the catalytic label is capable of catalysing a detectable change in the nucleic acid.

22. A method according to claim 21 wherein the catalytic label, or a component part thereof, is a recombinase, ligase, transposase, DNA polymerase, reverse transcriptase, or RNA polymerase.

23. A method according to any one of claims 1 to 14 wherein the catalytic label, or a component part thereof, is a kinase and the substrate label, or a component part thereof, is a molecule that is phosphorylated by the kinase.

24. A method according to any one of claims 1 to 14 wherein the catalytic label, or a component part thereof, is a phosphatase and the substrate label, or a component part thereof, is a molecule that is dephosphorylated by the phosphatase.

25. A method according to any one of claims 1 to 24 wherein the catalytic label is initially present in an inactive state and is subsequently activated.

26. A method according to claim 25 wherein the catalytic label is activated after the catalytic label and substrate label are brought into close proximity.

27. A method according to claim 25 or claim 26 wherein the catalytic label is activated by addition of an activator substance.

28. A method according to claim 25 or claim 26 wherein the catalytic label is activated by a change in pH.

29. A method according to claim 25 or claim 26 wherein the catalytic label is activated by a change in temperature.

30. A method according to claim 1 wherein step (b) is carried out under conditions which do not permit the catalytic label to act on the substrate label.

31. A method according to claim 2 wherein step (a) is carried out under conditions which do not permit the catalytic label to act on the substrate label.

32. A reagent kit comprising a first binding entity labelled with a catalytic label and a second binding entity labelled with a substrate label, characterised in that the catalytic label is capable of acting directly on the substrate label to generate a detectable change in the substrate label.

33. A reagent kit comprising a first interacting molecule labelled with a catalytic label and a second interacting molecule labelled with a substrate label, characterised in that the catalytic label is capable of acting directly on the substrate label to generate a detectable change in the substrate label.

34. A reagent labelling kit comprising a catalytic label and a substrate label, characterised in that the catalytic label is capable of acting directly on the substrate label to generate a detectable change in the substrate label, and means for attaching the catalytic label and substrate label to interacting molecules or to binding entities.


SEQUENCE LISTING (12 sequences)

SEQ ID NO: 1 - DNA, 54 bp, Artificial
  Oligonucleotide
cgggcctcttgcgggatatcgtccattccgacagcatcgccagtcactatggcg

SEQ ID NO: 2 - DNA, 50 bp, Artificial
  Oligonucleotide
atagtgactggcgatgctgtcggaatggacgatatcccgcaagaggcccg

SEQ ID NO: 3 - DNA, 56 bp, Artificial
  Oligonucleotide
tgctgctagcgctatatgcgttgatgcaatttctatgcgcacccgttctcggagca

SEQ ID NO: 4 - DNA, 60 bp, Artificial
  Oligonucleotide
tgctccgagaacgggtgcgcatagaaattgcatcaacgcatatagcgctagcagcacgcc

SEQ ID NO: 5 - DNA, 24 bp, Artificial
  322PCRAS Primer
acgggtgcgcatagaaattgcatc

SEQ ID NO: 6 - DNA, 24 bp, Artificial
  322PCRS Primer
gcgggatatcgtccattccgacag

SEQ ID NO: 7 - DNA, 54 bp, Artificial; identical to SEQ ID NO: 1
  Oligonucleotide

SEQ ID NO: 8 - DNA, 50 bp, Artificial; identical to SEQ ID NO: 2
  Oligonucleotide

SEQ ID NO: 9 - DNA, 56 bp, Artificial; identical to SEQ ID NO: 3
  Oligonucleotide

SEQ ID NO: 10 - DNA, 60 bp, Artificial; identical to SEQ ID NO: 4
  Oligonucleotide

SEQ ID NO: 11 - DNA, 24 bp, Artificial
  322PCRAS Primer
acgggtgcgcatagaaattgcatc

SEQ ID NO: 12 - DNA, 24 bp, Artificial
  322PCRS Primer
gcgggatatcgtccattccgacag